(19) United States
(12) Patent Application Publication
(10) Pub. No.: US 2010/0233131 A1
(43) Pub. Date: Sep. 16, 2010

(54) SELENIUM DEDIFFERENTIATED CELL, PREPARATION METHOD AND USAGE THEREOF

(75) Inventors: Soo Kyung Kang, Pusan (KR); Jeong Hwan Kim, Gimhae-si (KR)

Correspondence Address: GREENLEE WINNER AND SULLIVAN P C, 4875 PEARL EAST CIRCLE, SUITE 200, BOULDER, CO, 80301, US

(73) Assignee: PUSAN NATIONAL UNIVERSITY INDUSTRY-UNIVERSITY COOP, Pusan (KR)

(21) Appl. No.: 12/294,228

(22) PCT Filed: Jul. 31, 2008
(86) PCT No.: PCT/KR2008/004453

§ 371 Date: Sep. 23, 2008

(30) Foreign Application Priority Data
Jun. 26, 2008 (KR) 10-2008-0061126
Jul. 30, 2008 (KR) 10-2008-0074630

(51) Int. Cl. A61K 35/12; C12N 5/02; C12N 5/00; C01B 19/02

(52) U.S. Cl. 424/93.7; 435/377; 435/325; 423/510

(57) ABSTRACT

Provided are a cell therapeutic composition containing selenium, a method of dedifferentiating selenium-treated cells, a cell therapeutic composition containing cells dedifferentiated from the selenium-treated cells by the same method, and a cell therapeutic composition containing cells redifferentiated from the dedifferentiated cells.

The dedifferentiated cells, and cells redifferentiated therefrom, can be used to treat a variety of diseases.

### TECHNICAL FIELD

The present invention relates to a cell dedifferentiated by selenium, and more particularly, to a composition for cell dedifferentiation containing selenium, a method of dedifferentiating cells by selenium treatment, a dedifferentiated cell yielded by the method and a cell therapeutic composition containing the same, and a cell redifferentiated from the dedifferentiated cell and a cell therapeutic composition containing the same.

### BACKGROUND ART

Human embryonic stem cells can differentiate into all types of human cells and are expected to yield cures to a variety of diseases.

After embryo-derived human embryonic stem cells by Thomson Ph.D (USA) and his research group are first developed in 1998, several hundreds of human embryonic stem cell lines have been developed, and new embryo-derived human embryonic stem cell lines are still developing. Current human embryonic stem cell research is focused on feeder cells, establishing culture conditions excluding an animal-derived factor, and developing a differentiation technique with respect to specific cells.

However, due to limitations of embryonic stem cells, a clinical approach has been difficult so far. Embryonic stem cells are derived from blastocytes, from which self-derived embryonic stem cells cannot be obtained, and can trigger immune rejection when used therapeutically. Also, ethical controversy over embryo destruction is a reason to find alternatives to embryonic stem cells. To solve the problem of rejection by the immune system, research into stem cells customized by nuclear transfer is being conducted, but success has not yet been achieved. And, to avoid ethical controversy related to acquiring embryos, research into stem cells customized by dedifferentiation has been conducted.

Dedifferentiation technology has attracted attention in stem cell biology since it enables a somatic cell to be used in place of an embryonic stem cell, and thus is free from ethical controversy. Recently, stem cells dedifferentiated using the dedifferentiation technology, i.e., induced pluripotent stem cells (iPS cells), are attracting attention all over the world. The dedifferentiated stem cells are cells that are dedifferentiated into a pre-differentiated state by inserting a specific gene using somatic cells, and thus similarly serve as embryonic stem cells, which can differentiate into all types of human cells.

While many researchers have attempted to establish embryonic stem cells having the same type as the genotype of a patient from somatic cells by nuclear substitution or cell fusion technology, such efforts have not yet succeeded.

Meanwhile, Sinya Yamanaka, Ph.D (Japan) recently reported to produce dedifferentiated stem cells (iPS cells) from somatic cells using a four gene combination of genes specifically expressed in mouse embryonic stem cells, including Oct4, Sox2, KLF4 and c-Myc, and confirmed that they can be applied to human cells. Also, James Thomson Ph.D (USA) reported that human somatic cells can be dedifferentiated using another gene combination, including Oct4, Sox2, Nanog and Lin28.

The present inventors found that selenium-treated adipose tissue stromal cells isolated from an adipose tissue express stemness genes, exhibit increased cell proliferation, and have pluripotency to redifferentiate into various cell types, and these discoveries led them to the present invention.

### DISCLOSURE

### Technical Problem

The present invention is directed to providing a composition for cell dedifferentiation containing selenium.

The present invention is further directed to providing a method of dedifferentiating cells by selenium treatment.

The present invention is further directed to providing a dedifferentiated cell yielded by the above method and a cell therapeutic composition containing the same. The present invention is further directed to providing a cell redifferentiated from the dedifferentiated cell and a cell therapeutic composition containing the same.

### Technical Solution

The meaning of the term “dedifferentiation” is well known in the art. For example, it is disclosed in Weissman I. L., Cell 100: 157-168, FIG. 4 (2000). It means the regression of specialized, i.e., differentiated, mature somatic cells into a stem cell-like state to transfer or program to various cell types. It also means an increase in pluripotency, the number of cell types into which redifferentiation is possible.

In the present invention, cell dedifferentiation is induced by selenium. Selenium is an essential trace element for living organisms and a major component of an antioxidant enzyme that protects cells from free radicals generated in a normal oxygen metabolism, which indicates that selenium is safe for humans. In the present invention, organic selenium (e.g., selenomethionine or selenocysteine) or inorganic selenium (e.g., sodium selenite) may be used for cell dedifferentiation, but inorganic selenium is preferable.

In the present invention, cells subjected to dedifferentiation may be derived from mammals, preferably humans. Also, these cells may be isolated from a patient to be treated with cells dedifferentiated or redifferentiated therefrom. This enables the patient to undergo a desired treatment without immune rejection.

To be specific, the cells used herein may be derived from cumulus cells, skin, oral mucosa, blood, bone marrow, liver, lung, kidney, muscle, reproductive organ, or adipose tissue, all of which can be yielded from adult mammalian cells. For example, the cells include cumulus cells, epithelial cells, myofibroblast cells, neurons, keratinocytes, hematopoietic cells, melanocytes, chondrocytes, red blood cells, macrophages, monocytes, muscle cells, B lymphocytes, T lymphocytes, and adipose tissue stromal cells, but the present invention is not limited to these kinds of cells.

In the present invention, adipose tissue stromal cells are preferable for dedifferentiation. The adipose tissue stromal cells may be derived from an adipose tissue, which may be taken by any well-known method. For example, the adipose tissue can be taken from an abdominal region by liposuction. Such a method is remarkably easier than the conventional method using embryonic stem cells, and is free from ethical controversy.

The cells including adipose tissue stromal cells may be isolated from tissues taken by the conventional method. The tissue taken is microdissected to remove unnecessary parts, obtain a target cell, and isolate individual cells if possible. In one aspect, the microdissection of the tissue can be performed by physical means such as a homogenizer, a mortar, a blender, a scalpel, forceps or an ultrasonic device. In another aspect, the tissue can be dissected by an enzymatic method, for example, using serine protease, elastase or collagenase, but the present invention is not limited to these methods. In still another aspect, the tissue can be dissected by both mechanical and enzymatic methods.

The cells obtained in the previous step may be treated with selenium, or cultured for a specific period for proliferation and then treated with selenium, which is preferable.

The cells may be cultured in an appropriate medium under proper conditions depending on the species from which they originate. For example, mammalian cells may be cultured in a common medium for mammalian cells. The medium may be commercially available or prepared with components and percentages disclosed in the literature (e.g., catalog in American type culture collection). Commercially available media include Ham, IMDM Iscove's, Leibovitz L15, May Coy 5A, M199, Melnick's, MEM, NCTN, Puck's, RPMI, Swim S77, Trowell T8, Waymouth, Williams, DMEM and F12 media, but the present invention is not limited to use of these media. In the case of adipose tissue stromal cells, an α-MEM medium may be used.

Also, depending on the medium, serum (e.g., FBS), antibiotics (e.g., kanamycin, streptomycin, penicillin, etc.), growth factor (e.g., EGF, PDGF, VEGF, FGF, IGF, LIF, etc.), cytokine (e.g., insulin, estradiol, interleukin, corticosterone, etc.), or a trace element may be added. For adipose tissue stromal cells, 5 to 20% FBS may be added.

In a culture of adipose tissue stromal cells, when the cells have a confluency of 60 to 90%, and particularly 70 to 80%, they are treated with enzyme and subcultured. Here, the enzyme may be trypsin. In the present invention, the adipose tissue stromal cells are subcultured from 1 to 10 passages, preferably 2 to 5 passages, and more preferably, 3 passages.

Meanwhile, according to the present invention, before the selenium treatment, the cells may be starved to remove the influence of various components contained in serum during dedifferentiation. Then, the cells are further incubated in a medium containing serum at a concentration of 1 to 3%, and particularly 2%.

The present invention relates to a method of dedifferentiating cells by selenium treatment. Here, the selenium treatment comprises contacting the cells with selenium. For contacting with the cells, selenium may be directly added to an appropriate buffer solution, cell culture, or medium. In the present invention, the cells may be cultured in a selenium-containing medium.

Selenium is added at a concentration of 0.1 to 20 ng/ml, preferably 1 to 15 ng/ml, and more preferably 5 ng/ml, to the buffer solution, cell culture or medium containing cells to be dedifferentiated. When the concentration of selenium is more than 20 ng/ml, selenium induces cytotoxicity, and when the concentration of selenium is less than 0.1 ng/ml, dedifferentiation is not properly conducted. Culture time is 12 hours to 10 days, preferably 1 to 5 days, and more preferably 3 days. However, when the concentration of selenium is relatively lower, a treatment period has to be extended, and when the concentration of selenium is relatively higher, the treatment period is shortened to induce dedifferentiation, and thus the concentration of selenium and the culture time are not limited to the above values.

After the selenium treatment, the dedifferentiation of the cells induced by selenium is confirmed, and the dedifferentiated cells may be isolated from the buffer solution, medium or cell culture by a conventional method such as precipitation or centrifugation. The cells dedifferentiated by selenium and untreated cells (or differentiated cells) show a difference in expression of a specific gene. The selenium may induce 5%, preferably 10%, more preferably 20%, and even more preferably 30% or more, expression of the specific gene. The dedifferentiated cells have the following characteristics.

The dedifferentiated cells exhibit an increase in expression of a stemness gene compared to the differentiated cells. A ‘sternness gene’ is a gene that is significantly expressed in a stem cell. Stemness genes include Rexl, Nanog, Oct4, Sox2, Runx3, CDK1, CDK2, Nestin, VEGF and FGFR1. Also, the dedifferentiated cells exhibit an increase in expression of a cell growth-related factor, compared to the differentiated cells. The cell growth-related factor includes c-Myc, but the present invention is not limited thereto.

The dedifferentiated cells exhibit increased telomerase activity, and preferably about 2-fold increased telomerase activity. Also, compared to the differentiated cells, the dedifferentiated cells exhibit decreased expression of a specific gene but increased expression of a cell growth inhibiting gene (or tumor expansion inhibiting gene). The gene specifically expressed in the differentiated cells includes at least one of GFAP and Tuj, and the cell proliferation inhibition gene includes at least one of p53 and p31.

The dedifferentiated cells exhibit increases in PI3K expression and phosphorylation of its mediator, such as Rac, c-Raf, MEK, ERK, Stat3 or Akt, and inhibit expression of apoptosis-related protein, p-SAPK/JNK, induced by reactive oxygen species (ROS) compared to the differentiated cells.

The dedifferentiated cells exhibit lower stemness gene methylation on a promoter region than the differentiated cells. The stemness genes include at least one selected from the group consisting of Rex1, Nanog, Oct4 and Sox2.

The dedifferentiated cells exhibit increased expression of a cell migration-related gene compared to the differentiated cells. Thus, the cell migration is activated. The cell migration-related genes include at least one selected from the group consisting of MMP1, MMP3, SDF1, VEGF and CXCR4.

To analyze an expressed gene profile, RT-PCR, competitive RT-PCR, real time RT-PCR, RNase protection assay, Northern blot analysis or DNA chips can be used, but the present invention is not limited thereto. Also, Western blot analysis, ELISA, radioimmunoassay, Ouchterlony double immunodiffusion, Rocket immunoelectrophoresis, immunohistologic staining, immunoprecipitation assays, complement fixation, FACS or protein chips can be used, but the present invention is not limited thereto.

The present invention relates to a dedifferentiated cell yielded by the method of dedifferentiating cells by selenium treatment, and a cell therapeutic composition containing the same.

The dedifferentiated cell itself can be used to cure a disease. The dedifferentiated cells may redifferentiate into the above-mentioned types of cells in direct contact with a specific cell population in vivo. Thus, as the dedifferentiated cells are directly applied to a target tissue, there is no limit to the number of diseases that can be cured.

A method of producing a tissue using such a redifferentiated cell (Tissue Engineering) is well known in the art. Wang X has demonstrated that specific pancreatic cells can be converted into liver cells in fumaroy-laceto-acetate hydrolase (FAH)-deficient mice (Wang X. et al., “Liver Repopulation and Correction of Metabolic Liver Disease by Transplanted Adult Mouse Pancreatic Cells” Am. J. Pathol., 158(2):571-579). Lagasse demonstrated that hematopoietic stem cells obtained from bone marrow can be transplanted into FAH-deficient mice and then differentiate into hepatocytes (Lagasse et al., “Purified Hematopoietic Stem Cells Can Differentiate into Hepatocytes in Vivo,” Nature Medicine, 6(11); 1229-1234).

For in vivo redifferentiation of the dedifferentiated cells, the dedifferentiated cells may be injected, infused or transplanted into the specific cell population in vivo. Thus, the dedifferentiated cells may redifferentiate into the same type of cells in direct contact with the specific type cell population.

The dedifferentiated cells may be formed into a cellular composition including at least one diluent to protect and maintain the cells, and facilitate injection, infusion and transplantation into a target tissue. The diluent may include a buffer solution such as saline, phosphate buffered saline (PBS) or Hank's balanced salt solution (HBSS), serum or blood components.

The dedifferentiated cells may directly redifferentiate into a target cell type, or be stored in a medium for several days. In the latter case, to prevent loss of redifferentiation potential, cytokine or a leukemia inhibitory factor (LIF) may be added to the medium. Also, the redifferentiation capacity can be maintained by lyophilizing the cells.

The present invention relates to a cell redifferentiated from the dedifferentiated cells, and a cell therapeutic composition containing the same as an active component.

The dedifferentiated cells may redifferentiate into various cell types. The dedifferentiated cells may be redifferentiated into a specific cell type by a method well known in the art. For example, the methods can refer to Weissman I. L., Science 287:1442-1446 (2000), Insight Review Articles Nature 414: 92-131 (2000) and handbook “Methods of Tissue Engineering,” Eds. Atala, Al., Lanza, R. P., Academic Press, ISBN 0-12-436636-8; Library of Congress Catalog Card No. 200188747.

In addition, as described above, a specific cell type population is in contact with the dedifferentiated cells to redifferentiate into the specific cell type. Thus, when the dedifferentiated cells are in contact with a target specific cell type group, the dedifferentiated cells may redifferentiate into the target specific cell type.

Particularly, the dedifferentiated adipose tissue stromal cells may differentiate into mesodermal cells including osteocytes, chondrocytes and muscle cells, neurons, adipose cells or insulin-producing cells (B cells in pancreatic islet of Langerhans). The redifferentiated cells may be used to cure various diseases such as cancer, osteoporosis, arthritis, neurodegenerative disease, and diabetes.

The redifferentiation of the dedifferentiated adipose tissue stromal cells may be assayed by the following methods. The differentiation into osteocytes, chondrocytes and muscle cells may be detected by cell-specific staining. The differentiation into osteocytes and adipose cells may be detected by estimating generation of bone nodules and lipids. Also, the differentiation into osteocytes may be detected by estimating expression of AP and PPAR-γ, the differentiation into neurons may be analyzed by estimating expression of Tuj, GFAP, MAP2ab, and NF160, and the differentiation into insulin-producing cells may be detected by estimating expression of insulin.

The redifferentiated cells may be applied to a target tissue as described above for the dediffentiated cell to cure a disease. The redifferentiated cells may be injected, infused or transplanted into the target tissue. Also, a cell composition containing the dedifferentiated cells may include at least one diluent to protect and maintain the cells, and facilitate injection, infusion or transplantation into a target tissue.

### ADVANTAGEOUS EFFECTS

According to the present invention, dedifferentiated somatic cells capable of differentiating into various types of cells can be yielded by selenium treatment.

Particularly, the somatic cells including adipose tissue stromal cells are easily obtained and free from ethical controversy compared to conventional embryonic stem cells. Also, when the cells are obtained from a patient to be cured, they can exhibit desired therapeutic actions without immune rejection.

The dedifferentiated cells can be used to cure various diseases since they can differentiate into various types of cells.

### DESCRIPTION OF DRAWINGS

FIG. 1 shows proliferation efficiency of dedifferentiated adipose tissue stromal cells (ATSCs) according to the present invention.

FIG. 2 shows telomerase activity of dedifferentiated ATSCs according to the present invention.

FIG. 3 shows an increase in proliferation capability of dedifferentiated ATSCs according to the present invention.

FIG. 4 shows sternness gene expression in dedifferentiated ATSCs according to the present invention.

FIG. 5 shows cell proliferation stimulating or inhibiting gene expression in dedifferentiated ATSCs according to the present invention.

FIG. 6 shows activation of a growth-related signal in dedifferentiated ATSCs according to the present invention.

FIG. 7 shows influence of a p38 inhibitor on the growth of dedifferentiated ATSCs according to the present invention.

FIG. 8 shows influence of a MEK inhibitor on the growth of dedifferentiated ATSCs according to the present invention.

FIG. 9 shows a decrease in reactive oxygen species in cytoplasm by selenium.

FIG. 10 shows cell proliferation activity of ATSCs transfected with Rexl siRNA.

FIG. 11 shows a sternness gene, Rexl, plays a major role in dedifferentiation of ATSCs.

FIG. 12 is a schematic diagram showing a dedifferentiation mechanism for ATSCs by selenium.

FIG. 13 shows sternness gene methylation on a promoter region in dedifferentiated ATSCs according to the present invention.

FIG. 14 shows results of a cell migration assay for dedifferentiated ATSCs using a transwell membrane in vitro according to the present invention.

FIG. 15 shows results of a wound model assay for dedifferentiated ATSCs according to the present invention.

FIG. 16 shows cell migration-related gene expression in dedifferentiated ATSCs according to the present invention.

FIG. 17 shows analysis results for redifferentiation of dedifferentiated ATSCs into mesodermal cells according to the present invention

FIG. 18 shows in vivo analysis results for differentiation of dedifferentiated ATSCs into mesodermal cells according to the present invention.

FIG. 19 shows redifferentiation results for dedifferentiated ATSCs into neurons according to the present invention. (Se12: selenium 2 ng/ml, Se15: selenium 5 ng/ml)

### BEST MODE

Hereinafter, exemplary embodiments of the present invention will be described in detail. The present invention is not limited to the exemplary embodiments disclosed below, but can be implemented in various forms.

### Exemplary Embodiment 1

### Isolation and Culture of Adipose Tissue Stromal Cells and Selenium Treatment

To isolate adipose tissue stromal cells (ATSCs), raw adipose tissue samples were isolated from the human abdominal region in a clinic. The samples were washed with phosphate buffered saline (PBS), and digested at 37° C. for 30 minutes with 0.075% collagenase (Sigma, St. Louis, Mo., USA). After neutralization, the stromal cell pellets were collected via centrifugation and incubated overnight at 37° C. in a CO₂ incubator in a 10% FBS-containing α-MEM medium. The medium was replaced first after 48 hours of incubation, and then every 4^{th} day. When the confluency of the primary culture cells reached a confluency of 70 to 80% after 48 to 72 hours of incubation, the ATSCs were subcultured in 0.025% trypsin-containing solution.

For selenium treatment, the cultured ATSCs were seeded in 10 cm dishes at a density of 5×10⁵ and cultured in a 2% FBS-containing α-MEM medium for 8 hours at 37° C. in a CO₂ incubator. The cells were then treated with sodium selenite (Na₂SeO₃; sigma) at various concentrations for 3 days. The optimum concentration of selenium was determined on the basis of the results obtained from cytotoxicity studies using a broad concentration range for this reagent. Cell viability was evaluated via visual cell counts in conjunction with trypan blue exclusion. In all viability assays, triplicate wells were used for each condition, and each experiment was repeated at least three times.

### Exemplary Embodiment 2

### Induction of Dedifferentiation of ATSCs Via Selenium Treatment

### 2-1: Proliferation Capability of Dedifferentiated ATSCs Via Selenium Treatment

The proliferation of the ATSCs treated with various different concentrations of selenium (0, 5, 10, 15 and 20 ng/ml) for 3 days was evaluated via trypan blue exclusion.

A significant increase in proliferation efficiency of the ATSCs was assayed after treatment with 5 ng/ml selenium for 3 days.

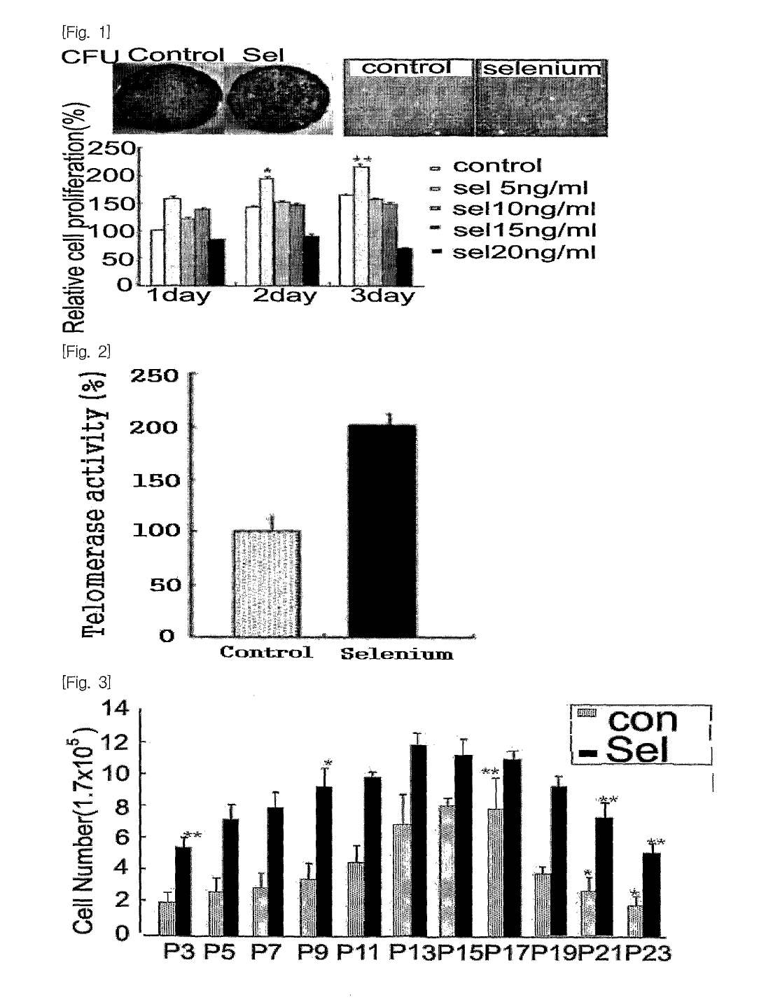

Also, the proliferation efficiency of colony forming units (CFU) in the selenium-treated cells was evaluated. The CFU is a population derived from a single cell, an increase of which indicates that selenium actively stimulated the proliferation of the ATSCs. First, the ATSCs were seeded in 10 cm dishes at a density of 5×10⁵ and cultured in a 2% FBS-containing α-MEM medium for 8 hours at 37° C. in a CO₂ incubator. The cells were then treated with selenium (5 ng/m1) for 3 days. For the CFU assay, control ATSCs (not treated with selenium) and the selenium-treated ATSCs were seeded in 10 cm dishes at a density of 2×10² and cultured in a 10% FBS-containing α-MEM medium at 37° C. in a CO₂ incubator. After 15 days, the cells were fixed with 4% paraformaldehyde (PFA) for 30 minutes at room temperature and stained with 0.1% toluidine blue dissolved in 1% PFA. The proliferation efficiency of the CFU was evaluated via visual colony counts. CFU levels showed that cell proliferation in the selenium-treated ATSCs increased at least 1.8 times (FIG. 1).

### 2-2: Induction of Dedifferentiation of ATSCs Via Selenium Treatment

To analyze the effects of selenium on reactive oxygen species (ROS) scavenging from the ATSCs, 5 ng/ml of selenium was treated for 3 days, and then telomerase activity was measured. An increase in telomerase activity is a characteristic of a stem cell, and a decrease in telomerase activity indicates differentiation of the cells.

Selenium-treated ATSCs have a 2-fold increase in telomerase activity, according to which it can be noted that the selenium induces dedifferentiation of the cells (FIG. 2).

### 2-3: Increase in Proliferation Efficiency of ATSCs Via Selenium Treatment

During prolonged culture periods, the population of control ATSCs underwent a progressive reduction in proliferation potential. The cells finally underwent senescence after 21 to 23 passages (90 to 100 days in culture). At the end of the proliferation lifespan, the cells were flattered and larger in morphology in a monolayer similar to that described for senescent fibroblasts. In experimental selenium exposure, selenium-treated ATSCs grew continuously for more than 3 months (>21 passages; FIG. 3). The rate of proliferation of selenium-treated ATSCs resembled that of the control ATSCs. In addition, the ATSCs exposed to selenium retained their inhibition for cellular proliferation via cell-to-cell contact. The results show that the extended growth of stromal cells, as a consequence of selenium exposure, did not alter cell growth properties.

### 2-4: Increase in Stemness Gene Expression in ATSCs Via Selenium Treatment

The expression of molecular markers in both the control ATSCs and the selenium-treated cells was verified via real time RT-PCR and Western blot analysis.

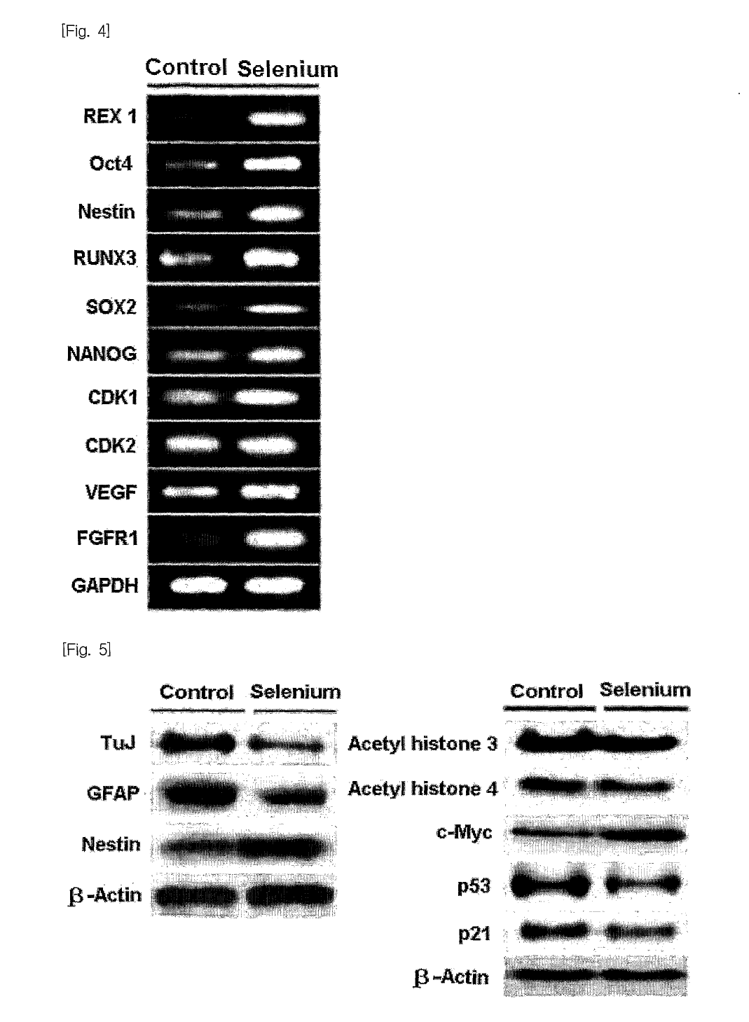

As shown in FIG. 4, the selenium treatment induced overexpression of several stemness genes and functional genes (Rex1, Nanog, Oct4, Sox2, Runx3, CDK1, CDK2, Nestin, VEGF and FGFR1). Particularly, Rexl expression was significantly increased as a result of selenium treatment.

In addition, as shown in FIG. 5, it was confirmed that the selenium treatment induced expression of Nestin and c-Myc, and downregulation of GFAP, Tuj, p53 and p21. Additionally, the selenium treatment attenuated acetylation of Histones 3 and 4. This result shows that the selenium treatment induces dedifferentiation of the ATSCs.

### Exemplary Embodiment 3

### Induction of Growth-Related Signal in ATSCs Dedifferentiated by Selenium

### 3-1: Relevance of Growth-Related Signaling Pathway in ATSCs Dedifferentiated by Selenium

To identify activated signaling molecules related to cell proliferation occurring after the selenium treatment, total protein levels and phosphorylation status of several proliferation-related proteins were analyzed via Western blot analysis.

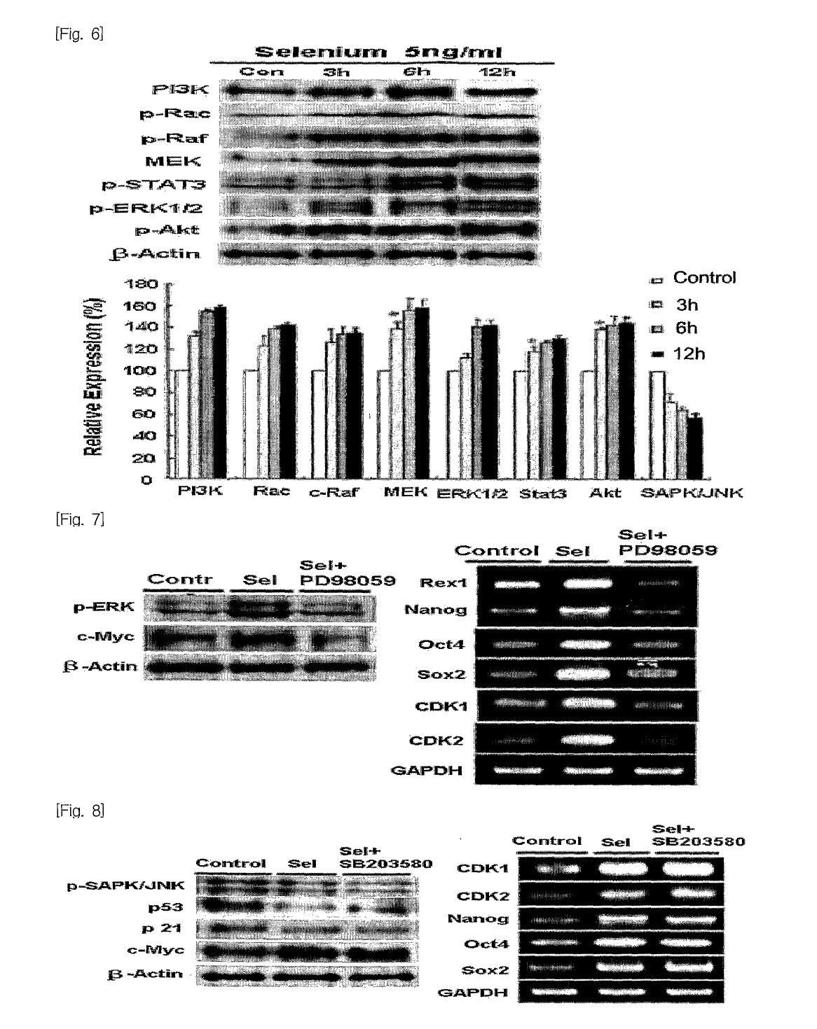

FIG. 6 shows the Western blot results in selenium-treated ATSCs for different lengths of time (0, 3, 6 and 12 hours). Selenium induced significant activation of PI3K and its downstream mediators (p-Rac, p-c-Raf, p-MEK, p-ERK, p-Stat3 and p-Akt) in a time-dependant manner. However, the selenium treatment reduced the concentration of apoptosis-related protein, p-SAPK/JNK, in a time-dependant manner.

### 3-2: Relevance of p38 and MEK Signaling Pathways in Control of Cell Growth of ATSCs Dedifferentiated by Selenium

To confirm the relevance of p38 and MEK signaling pathways in control of cell growth of the selenium-treated ATSCs, the selenium-treated ATSCs were treated with SB203580 (10 μM; p38 inhibitor) and PD98059 (10 μM; MEK inhibitor), and then analyzed via Western blot analysis and RT-PCR.

As shown in FIG. 7, the results of Western blot analysis indicated that SB203580 induced downregulation of p-SARK/JNK and p53 and p21 proteins, and overexpression of c-Myc protein. Also, the RT-PCR results show that SB203580 upregulated proliferation-related transcription factors including CDK1 and CDK2.

These data indicated that selenium can directly reduce the levels of the apoptosis-related protein, p-SAPK/JNK. As shown in FIG. 8, PD98059 downregulated p-ERK and c-Myc proteins. Also, the RT-PCR results show that PD98059 downregulated proliferation-related transcription factors including Rex1 and CDK1. These data clearly show that selenium directly inhibits apoptosis-related proteins, p-SAPK/JNK, and induces the proliferation of ATSCs via the activation of the MEK and PI3K signaling pathways.

### 3-3: ROS Generation in ATSCs Dedifferentiated by Selenium

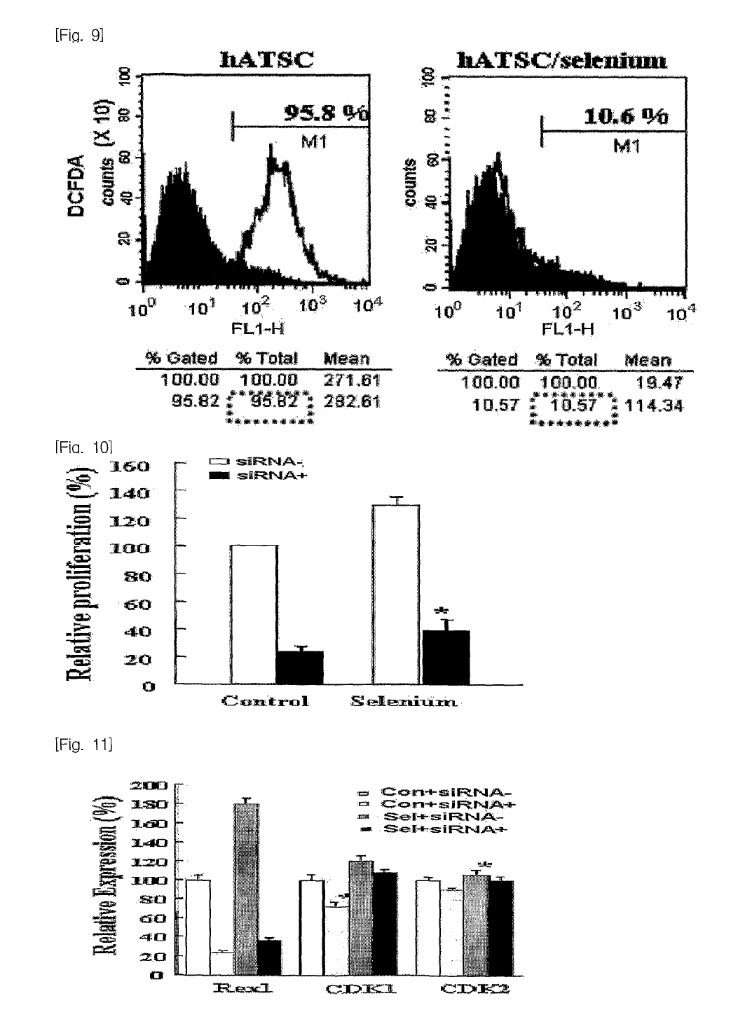

Selenium regulation to ROS generation was evaluated. The ROS generation from the ATSCs increased oxidation of 2′, 7′-dichlorodihydrofluorescein (DCF) in a concentration-dependant manner, and the increased florescence intensity of DCF was compensated by selenium treatment (FIG. 9). These results show that selenium induced the proliferation of ATSCs on the result of the activation of MEK and PI3K signaling pathways and inhibition of ROS generation.

### 3-4: Relevance of Rex1 to Proliferation of ATSCs Dedifferentiated by Selenium

ERK1/2 and Akt activation in the selenium-treated ATSCs resulted in the induction of stemness transcription factor expression, and particularly, Rex1 expression. In order to evaluate the roles of Rexl in the proliferation of selenium-treated ATSCs, the ATSCs were transfected with Rex1 silencing siRNA prior to selenium treatment. Rex1 siRNA-transfected cells were harvested and examined via measurements of cell proliferation activity (FIG. 10) and changes in the expression of Rex1, CDK1 and CDK2 mRNA (FIG. 11). As shown in FIGS. 10, 11 and 3B, the Rex1 siRNA-transfected cells were significantly inhibited in cell growth and Rexl gene expression compared to the untreated controls. These results show that Rexl is a major gene closely associated with the ATSC proliferation, and selenium increases proliferation efficiency of selenium-treated ATSCs by the enhancement of Rex1 expression.

Based on these results, a model for explaining mechanisms of proliferation and dedifferentiation of ATSCs induced by selenium treatment was suggested (FIG. 12).

### Exemplary Embodiment 4

### Induction of Epigenetic Reprogramming in Selenium-Treated Dedifferentiated ATSCs Via DNA Demethylation

### 4-1: Change in Gene Expression Pattern

To confirm the pattern of gene expression, oligonucleotide microarray analysis was performed. The analysis of gene expression levels indicated that less than 6% of the total genes exhibited a more than 2.2-fold difference in expression level in the control ATSCs and the selenium-treated ATSCs, as shown in y level (=0.89). Compared with the control ATSCs, the selenium-treated ATSCs exhibited upregulation of the cell proliferation-associated genes (42%).

### 4-2: Change in DNA Methylation on Rex1 and Nanog Promoter Regions

To determine whether selenium treatment was capable of inducing epigenetic modifications on exogeneous chromatin templates, changes in DNA methylation on Rex1 and Nanog promoter regions were analyzed. Also, a bisullipide sequencing analysis was performed in order to establish 5′-3′ CpG methylation profiles across a proximal promoter, a proximal enhancer and an early transcription start site (TSS) of each gene.

Genomic DNA of ATSCs was purified by phenol/chloroform/isoamylalcohol extraction, and chloroform extraction once, and then was precipitated with ethanol. The DNA was dissolved in distilled water. Bisullipide conversion was performed using EZ DNA Methylation-Gold Kit (Zymo Research, USA), as described by a manufacturer. That is, unmethylated cytosines on the DNA were converted into uracils via the heat-denaturation of DNA with specifically designed CT conversion reagent. The DNA was then desulphonated and subsequently cleaned and eluted. The bisulfite-modified DNA was then immediately used for PCR or stored at −20° C. or below. The converted DNA was amplified via PCR using primers designed with MethPrimer (http://www.urogene.org/methprimer). The PRC was conducted in a MyGenie 96 Gradient Thermal Block (Bioneer, Daejeon, South Korea) according to the following protocol, including 95° C. for 15 minutes, 40 cycles of 95° C. for 20 seconds, 43 to 58° C. for 40 seconds, and 72° C. for 30 seconds, elongation at 72° C. for 10 minutes and soaking at 4° C. After electrophoresis on 1.5% agarose gel, the remaining PCR products were cloned into bacteria (DH5α) by the pGEM T-Easy Vector System I (Promega, Madison, Wis., USA). The DNA extracted from bacterial clones were analyzed via sequencing with M13 reverse primers using the ABI 3730XL capillary DNA sequencer (Applied Biosystems, Foster City, Calif., USA), and represented as rows of circles, each circle denoting the methylation status of CpG.

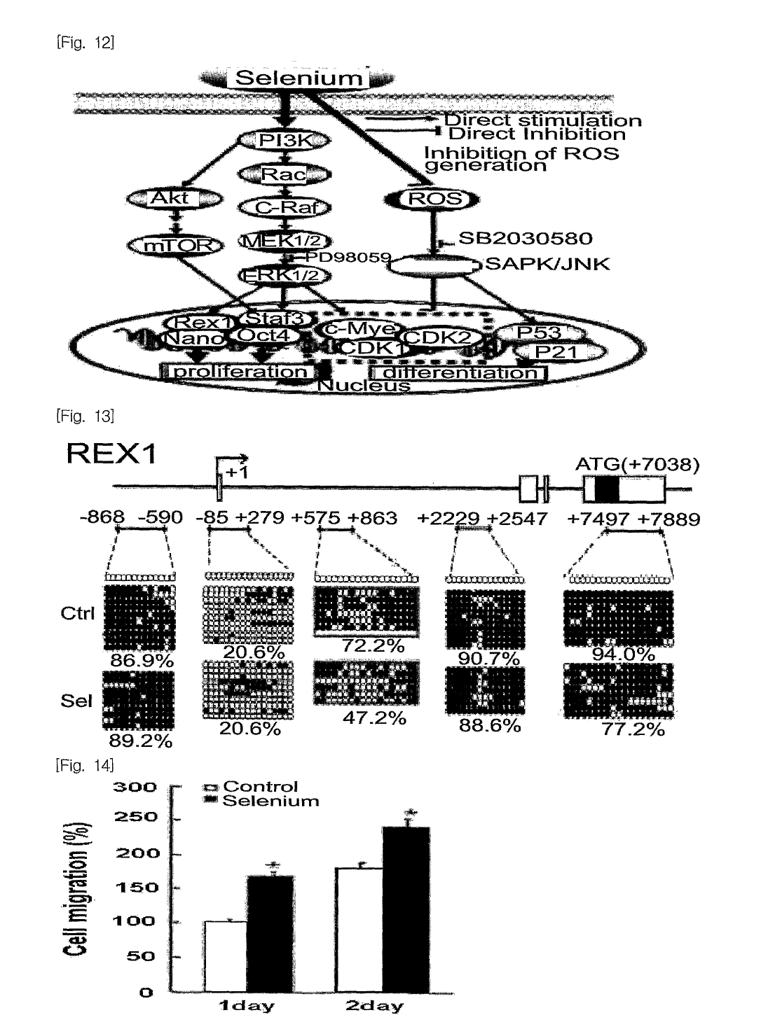

As shown in FIG. 13, in the Rex 1 region, 5 amplicons were analyzed, which collectively converted strongly methylated CpG dinucleotides within nucleotides −868 to +7889 relative to the TSS. The Rex 1 region was methylated in the control

ATSCs, and significantly demethylated from 72.2% (control ATSCs) to 42.2% (selenium-treated ATSCs) in the third region. Three regions were evaluated in the Nanog promoter, which was effectively demethylated in the third region (−86 to +66) to the TSS. Three regions were also evaluated in the Oct4 promoter, and included CpG in nucleotides −57 to +66 to the TSS. This Oct4 methylation pattern was downregulated in the selenium-treated ATSCs (third region; 30.3%) compared to the control ATSCs (third region; 65.7%).

### Exemplary Embodiment 5

### Induction of Increase in Migration Activity of ATSCs Dedifferentiated by Selenium

### 5-1: Cell Migration Assay Using Transwell Membrane In Vitro

To estimate a migration activity of selenium-treated ATSCs, cells were seeded in 10 cm dishes at a density of 5×10⁵, and cultured in a 2% FBS-containing α-MEM medium at 37° C. for 8 hours in a CO₂ incubator. The cells were then treated with selenium (5 ng/ml) for 3 days. The cultured cells were transferred into Costar transwell membrane (8 μm pore size), and placed on 6-well plates. Under the membrane, selenium and a 2% FBS-containing α-MEM medium were added to each well. In an upper chamber, the cells were incubated in a 2% FBS-containing α-MEM medium at 37° C. for 2 hours, and the plates were incubated overnight at 37° C. in a CO₂ incubator. The cells on the lower surface of the plate were dried, counterstained with Harris hematoxylin for 20 minutes, and then washed. The stained inserts were placed on an object slide, and the number of cells was counted under a 200× inverted bright field microscope. The cell counts were repeatedly estimated to ten bright fields under the 200× microscope, and their average was calculated. The migration was represented as the count of cells per field of the spontaneous migration which was non-specifically determined.

As shown in FIG. 14, the selenium-treated ATSCs exhibited significantly increased migration efficiency compared to the control ATSCs in a time-dependant manner.

### 5-2: Wound Model Assay

To obtain clear evidence for the role of selenium in ATSC migration, a simple cell scraped wound model assay was performed. Cells were seeded into 60 mm culture dishes, and a straight line was lightly carved across the center, outer and bottom surface of each dish with a scalpel. The ATSCs were incubated overnight in a serum-free medium, scraped from one side of the marked line, and washed three times with a medium to remove loose or dead cells. Subsequently, the cells were stimulated with 5 ng/ml of selenium, and incubated at 37° C. for 24 hours. The control dish was also scratched in the same manner as described above, and incubated in a selenium-free medium. Cells migrated across the marked reference line were photographed under a phase contrast microscope.

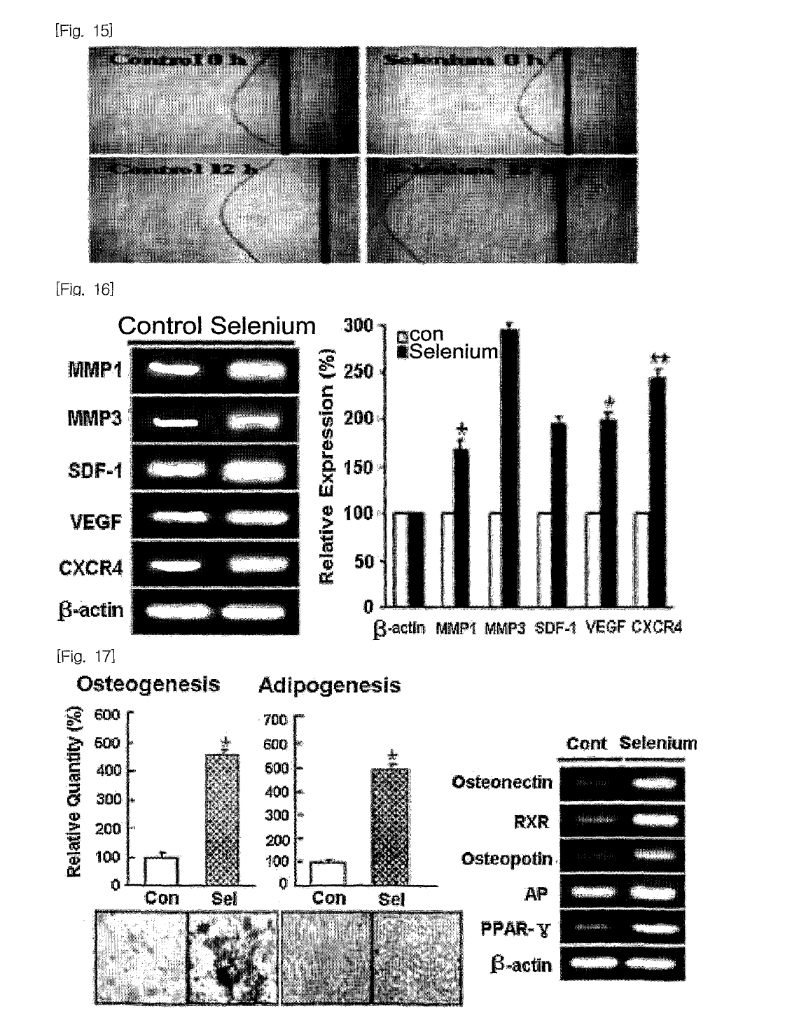

As shown in FIG. 15, selenium increased migration of ATSCs across the reference line to three times higher than that of the control ATSCs. These results correspond to increases in expression of transcription factors associated with migration, MMP1, MMP3, SDF1, VEGF and CXCR4, after the selenium treatment (FIG. 16).

### MODES OF THE INVENTION

### Exemplary Embodiment 6

### Differentiation Potential of ATSCs Dedifferentiated by Selenium

6-1: Differentiation Potential into Mesoderm-Like Cells in Selenium-Treated Dedifferentiated ATSCs In Vitro

To estimate the differentiation potential of selenium-treated ATSCs, osteogenic and adipogenic differentiation potential was evaluated.

In the present embodiment, it was confirmed that the selenium-treated ATSCs accumulated significant amounts of calcium and lipid droplets after only one week of osteogenetic and adipogenetic induction in vitro (FIG. 17). Differences in formation of bone nodules and lipid droplets between the control and selenium-treated ATSCs were estimated by calculating the number of stained bone nodules and lipid droplets in 25 randomly selected fields (regions). As shown in FIG. 17, von Kossa-positive staining for calcium precipitations and Nile red staining for lipid droplets were observed in the selenium-treated ATSCs. The selenium-treated ATSCs exhibited significant calcium accumulation and lipid formation. Five-fold more nodules and six-fold more lipid droplets than the control ATSCs were observed in the selenium-treated ATSCs by an eluted dye quantitative assay using a spectrophotometer. These results corresponded to overexpression of osteogenesis- and adipogenesis-related transcription factors including osteonectin, RXR, osteopontin, AP and PPAR-γ after the selenium treatment (FIG. 17). The selenium-treated ATSCs induced significant increases in the level of osteonectin, RXR, and osteopontin mRNA during osteogenesis. Also, the selenium-treated ATSCs induced significant increases in levels of AP and PPAR-γ mRNAs in adipogenesis (FIG. 17).

6-2: Differentiation Potential into Mesoderm-Like Cells in Selenium-Treated Differentiated ATSCs In Vivo

In vivo osteogenesis and chodrogenesis effects were evaluated. To this end, the control ATSCs and selenium-treated ATSCs were fixed with Matrigel (BD Bioscience, San Jose, Calif., USA). About 2×10⁶ cells were mixed with Matrigel, which were subcutaneously transplanted to 6-week-old immunodeficient beige mice (NIH III/bg/nu/xid; Charles River Laboratories, Wilmington, Mass., USA). The procedures were conducted according to an approved protocol. Transplants were recovered at the sixth week after the transplantation, fixed with 4% formalin, and then decalcified with 10% EDTA (pH 8.0) for paraffin embedding. The paraffin-embedded sections were deparaffinated and stained with Alizarin Red (bone), Masson (muscle and chondrocytes) and Van Gieson (chondrocytes) stains.

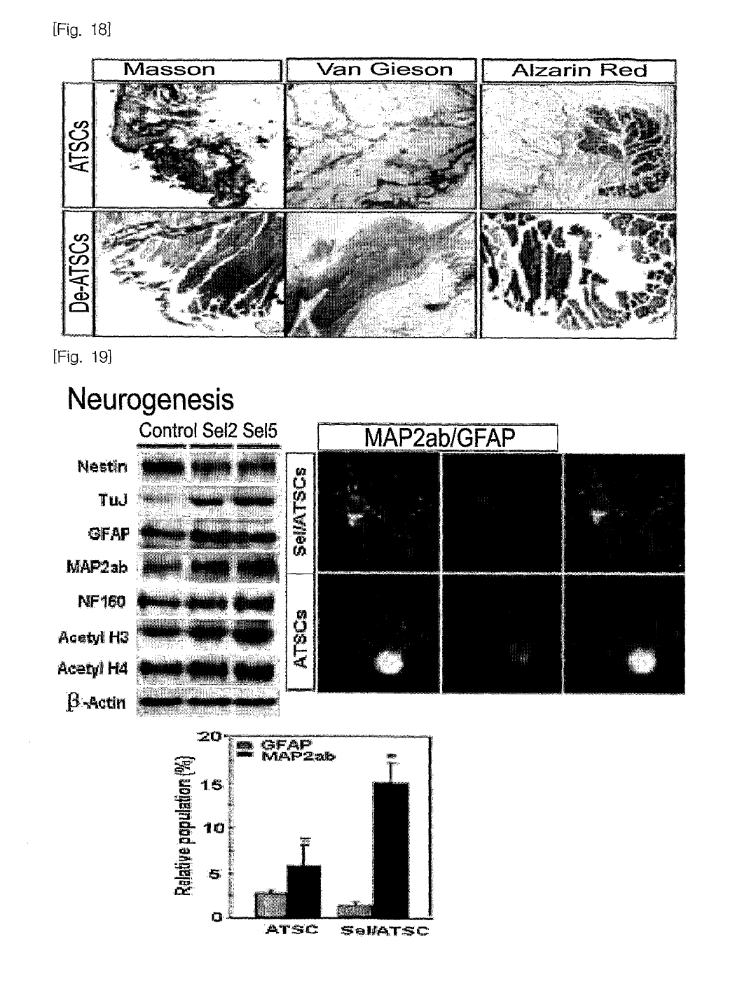

As shown in FIG. 18, the results of Alizarin red staining for the transplanted tissues showed an increase in osteogenesis in the selenium-treated ATSCs compared to the control ATSCs. The control ATSC-transplanted tissue sections failed to show effective bone and collagen fiber staining. Intensive Masson staining also showed that the selenium-treated ATSC transplants were effectively differentiated into muscle fibers.

6-3: Differentiation Potential of Selenium-Treated Dedifferentiated ATSCs into Neurons and Insulin-Producing Cells

In an attempt to estimate differentiation potential of selenium-treated ATSCs into neural cells in vitro, low levels of Nestin protein expression were detected in the selenium-treated ATSCs after the induction of differentiation (FIG. 19). After neural differentiation, the selenium-treated ATSCs expressed higher levels of Tuj, GFAP, MAP2ab and NF160 than the control ATSCs (FIG. 19). The control ATSCs did not undergo efficient neural differentiation under the conditions applied in the present embodiment. It means that there is a difference in differential potential between the control ATSCs and the selenium-treated ATSCs. The results of Western blot analysis show that selenium-treated ATSCs overexpressed acetyl-histones 3 and 4 after the neural differentiation (FIG. 19).

According to the immunocytochemical data, neuroprogenitors (neurospheres) can be expanded with bFGF, EGF and BDNF, and more extensive differentiation is caused by the removal of cytokines and growth on PDL-laminin-coated surfaces (FIG. 19). Populations of differentiated selenium-treated ATSCs exhibited morphological and phenotypic characteristics corresponding to astrocytes (GFAP) and neurons (MAP2ab) after induction of differentiation in vitro. Compared with the control ATSCs (2.4% of total cells), a high percentage of neural differentiation (MAP2ab/total cells) was detected in the selenium-treated ATSCs (9.0% of total cells; FIG. 19). As the results of postnatal mouse brain transplantation, considerable numbers of the selenium-treated ATSCs were inserted into the hippocampus, striatum and cortex, and they effectively differentiated into MAP2ab-positive neurons in the hippocampus (FIG. 19).

Moreover, for differentiation of cells similar to beta cells, cells were cultured in “N2 media-NA” containing DMEM/F12 (Gibco-Invitrogen) supplemented with 10 mM nicotinamide, ITS (1:50), B27 (1:50; Invitrogen) and 15% FBS. After 24-hour culture, the medium was changed with a high glucose (3500 mg/L)-containing differentiation medium for 2 weeks. After the induction of differentiation, double immunocytochemistry was conducted using insulin (1:800; Sigma) and c-peptide (1:100; Milipore) antibodies.

According to the present invention, the selenium-treated ATSCs effectively dedifferentiated into insulin-secreting cells. Differentiated selenium-treated ATSCs secreted a significant amount of insulin with C-peptide in contrast to differentiated control ATSCs.

### INDUSTRIAL APPLICABILITY

According to the present invention, using selenium, which is safe for humans, cells including ATSCs may be dedifferentiated, and then the dedifferentiated cells, and cells redifferentiated therefrom, may be used to cure various diseases.

1. A composition for cell differentiation containing selenium.

2. The composition according to claim 1, wherein the cells are isolated from mammals.

3. The composition according to claim 2, wherein the cells are adipose tissue stromal cells.

4. A method of dedifferentiating cells, comprising:
treating cells with selenium.

5. The method according to claim 4, further comprising:
culturing the cells in a 1 to 3% FBS-containing medium before the selenium treatment.

6. The method according to claim 4, wherein the selenium is treated at a concentration of 0.1 to 20 ng/ml for 12 hours to 10 days.

7. The method according to claim 4, wherein the cells are isolated from mammals.

8. The method according to claim 7, wherein the cells are adipose tissue stromal cells.

9. The method according to claim 7, wherein the differentiated cells exhibit an increase in expression of a stemness gene selected from the group consisting of REX1, Nanog, Oct4, Sox2, Runx3, CDK1, CDK2, Nestin, VEGF and FGFR1, compared to the differentiated cells.

10. The method according to claim 7, wherein the dedifferentiated cells exhibit an increase in c-Myc expression compared to differentiated cells.

11. The method according to claim 7, wherein the dedifferentiated cells exhibit an increase in telomerase activity compared to differentiated cells.

12. The method according to claim 7, wherein the dedifferentiated cells exhibit a decrease in GFAP and Tuj gene expression compared to differentiated cells.

13. The method according to claim 7, wherein the dedifferentiated cells exhibit a decrease in p53 and p31 expression of compared to differentiated cells.

14. The method according to claim 7, wherein the dedifferentiated cells exhibit increases in PI3K gene expression, and phosphorylation for a mediator of the PI3K gene selected from the group consisting of Rac, c-Raf, MEK, ERK, Stat3 and Aid, compared to differentiated cells.

15. The method according to claim 7, wherein the dedifferentiated cells exhibit a decrease in p-SAPK/JNK gene expression compared to differentiated cells.

16. The method according to claim 7, wherein the dedifferentiated cells exhibit a decrease in methylation of a stemness gene selected from the group consisting of REX1, Nanog, Oct4 and Sox2, on a promoter region, compared to differentiated cells.

17. The method according to claim 7, wherein the dedifferentiated cells exhibit an increase in expression of a cell migration-related gene selected from the group consisting of MMP1, MMP3, SDF1, VEGF and CXCR4, compared to differentiated cells.

18. A dedifferentiated cell yielded by the method according to any one of claims 4 to 17.

19. A cell therapeutic composition containing the dedifferentiated cells according to claim 18 as an active component.

20. A cell redifferentiated from the dedifferentiated cell according to claim 18.

21. The cell according to claim 20, wherein the redifferentiated cell is selected from the group consisting of a mesodermal cell, a neuron, an adipose cell and an insulin-producing cell.

22. A cell therapeutic composition containing the redifferentiated cell according to claim 20 or 21 as an active component.
